(19) United States
(12) Patent Application Publication
(10) Pub. No.: US 2005/0239899 A1
(43) Pub. Date: Oct. 27, 2005

(54) Beta-secretase inhibitors

(76) Inventors: Wolfgang Fecke, Florence (IT); Marcus Hammerle, Berlin (DE); Ulrich Rester, Wuppertal (DE)

Correspondence Address: SUTHERLAND ASBILL & BRENNAN LLP, 999 PEACHTREE STREET, N.E., ATLANTA, GA, 30309, US

(21) Appl. No.: 10/502,075

(22) PCT Filed: Jan. 20, 2003
(86) PCT No.: PCT/EP03/00504

§ 371 Date: Apr. 18, 2005

(30) Foreign Application Priority Data
Jan. 18, 2002 (EP) 02001339.7
Jun. 5, 2002 (EP) 02012566.2

(51) Int. Cl. A61K 31/15

(52) U.S. Cl. 514/639

(57) ABSTRACT

The invention relates to novel beta-secretase inhibitors.

The present invention relates to novel inhibitors of the aspartyl protease BACE (beta-secretase), to their pharmaceutical compositions and to their use for treating diseases caused by amyloid beta peptide depositions such as Alzheimer disease and Down Syndrome.

Alzheimer's disease (AD) is a neurodegenerative disorder clinically characterized by progressive dementia that inevitably leads to incapacitation and death. Upon autopsy, massive synaptic loss and neuronal death is observed in brain regions critical for cognitive function, including cerebral cortex, entorhinal cortex, and hippocampus (reviewed R. D. Terry, E. Masliah, L. A. Hansen, The neuropathology of Alzheimer disease and the structural basis of its cognitive alterations, in: R. D. Terry et al. (Ed.), Alzheimer Disease, Lippincott, Williams and Wilkins, Philadelphia, 1999, pp. 87-206). The inexorable loss of neurons and synapses over the course of AD is responsible for the dementia that slowly robs AD patients of their memories, personalities, and eventually their lives.

Two characteristic brain lesions define Alzheimer's Diseases at the microscopic level: neurofibrillary tangles and beta amyloid (or neuritic) plaques. Neuritic plaques surrounded by neuronal injury are found in brains of all patients suffering from AD. The main component of these plaques is the 42 amino acid form of the amyloid-beta peptide (A beta). This peptide is neurotoxic and easily forms insoluble fibrils that aggregate into plaques. The accumulation of the A beta peptide is not only a hallmark of AD but also characterizes the brains of individuals with Trisomy 21 (Down's Syndrom), Hereditary Cerebral Hemorrhage with Amyloidosis of the Dutch Type (HCHWA-D), and other neurodegenerative disorders.

The 39-42 amino acids A beta peptide is generated by proteolysis of the amyloid precursor protein (APP). Several proteases called secretases are involved in the processing of APP.

Cleavage of APP at the N-terminus of the A beta peptide by beta-secretase and at the C-terminus by gamma-secretase constitutes the beta-amyloidogenic pathway, i.e. the pathway by which A beta is formed. A description of the proteolytic processing fragments of APP is found, for example in Citron M., Neurobiology of Aging 23 (2002), 1017-1022.

The aspartyl protease responsible for processing of APP at the beta-secretase cleavage site was recently identified by (Vassar R. et al., Science (1999) 286, 735-741.). This beta-secretase has been disclosed using various nomenclature, including beta amyloid converting enzyme 1 (BACE1), Asp 2 and Memapsin 2. Importantly, BACE1 knockout mice fail to produce A beta, and present a normal phenotype. When crossed with transgenic mice that overexpress APP, the progeny show reduced amounts of A beta in brain extracts as compared with control animals (Luo et al., 2001, Nature Neuroscience 4: 231-232). This evidence strongly supports the proposal that inhibition of beta-secretase activity and reduction of A beta in the brain provides a therapeutic method for treatment of AD and other beta amyloid disorders.

At present there are no effective treatments of halting, preventing, or reversing the progression of Alzheimer's disease. The current therapeutics for AD are all cholinergic agents; specifically, inhibitors of acetycholinesterase (ACHE). The basis for this approach is the fact that AD causes substantial loss of cholinergic neurons. ACHE inhibitors increase the levels of acetylcholine to keep the remaining cholinergic neurons firing.

Unfortunately, this type of therapy does not stop the progressive loss of cholinergic neurons, and eventually becomes ineffective. Moreover, several neurotransmitter systems are altered in AD. A better approach would be to develop agents that affect the molecules that are responsible for the neurodegeneration. Major efforts have been made to block A beta-production and aggregation in the brain by targeting the alpha, beta or gamma secretases (See for example, Sabbagh, M. et al., Alz. Dis. Rev. (1997) 3, 1-19). However, BACE-1 appears to be the optimal therapeutic target because (I) it catalyzes the initial, rate limiting step in A beta production, and (II) BACE-1 knockout mice do not show any apparent phenotype.

Among the few reported inhibitors of Beta-secretase so far are substrate-based, transition state analogues. PCT application WO 01/00665 C2 entitled “Catalytically active memapsin and methods of use thereof” describes the substrate specificity of the BACE enzyme, the first peptidomimetic inhibitors (OM99-1 and OM99-2) and the crystal structure of the inhibitors complexed with the enzyme. US20020115616 entitled “Novel inhibitors of Beta Amyloid Cleavage Enzymes” also describes peptidomimetic compounds. Despite their potency, these compounds are relatively large and show poor ability to cross biological membranes. For agents to work effectively in vivo, the compounds must not only cross the blood-brain barrier, but they must also be taken up by cells. As they must work inside the cell, these agents should be highly selective: interference with other intracellular proteases and critical signaling pathways must be minimized.

Further BACE inhibitors are described in WO 02/08810, WO 02/02520, WO 02/02518, WO 02/02512, WO 02/02506, WO 02/02505, WO 02/76440 and WO 02/47671.

However, since Alzheimer's disease is a wide-spread disease, with about 4 million people suffering therefrom in the U.S. alone, there is a great need for effective substances to treat this disease.

Therefore, it was an object of the invention to provide effective beta-secretase inhibitors which should further be able to cross biological membranes.

According to the invention this object is achieved by a beta-secretase inhibitor of formula 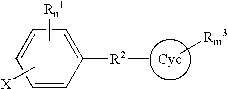
wherein
X: represents a halogen or a moiety which is bioisosteric thereto, in particular, F, Cl, Br, I, Methyl or CF₃, preferably Cl.
R1: each independently represents halogen, hydroxy, cyano, trifluoromethyl, nitro, a hydrocarbon group containing 1 to 4 carbon atoms, in particular, C1-C4 alkyl, C2-C4 alkenyl or C2-C4 alkynyl, which may be substituted, e.g. hydroxyalkyl, haloalkyl, cyanoalkyl, carboxyalkyl, acylalkyl, oxyalkyl, sulfonylalkyl, sulfonylamidoalkyl, amidoalkyl, carbonoylalkyl, ureylalkyl, etc. or a moiety which is bioisosteric thereto and n=0 to 4, preferably n=0 to 2.
R2: is a connecting moiety from a group consisting of a single bond or a C1-C8 hydrocarbon group, in particular, a C1-C4 alkylene group, a C2-C8 alkenylene group, a C2-C8 alkynylene group, a C1-C4 alkylene group containing at least one heteroatom, a C2-C8 alkenylene group containing at least one heteroatom or a C2-C8 alkynylene group containing at least one heteroatom.
Cyc: is a carbocyclic, aryl or heterocyclic moiety.
R3: each independently is a group being bound to the moiety Cyc and is selected from R1 or is a aryl or heterocyclic moiety substituted by 0 to 4 moieties from R1 or a group selected from 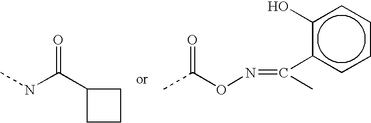
   and m=0 to 8, in particular 0 to 4.

The beta-secretase inhibitors of the invention are characterized by the presence of a halophenyl group, in particular, a chlorophenyl group, whereby a parachlorophenyl group, a diorthochlorophenyl group as well as a dimetachlorophenyl group are preferred. The phenyl group can be further substituted, e.g. with an OH group, with a dimetachloro-ortho-hydroxy-phenyl group being preferred.

The group X can be in ortho, meta or para position.

R¹ preferably is C1-C4 alkyl, C2-C4 alkenyl or C2-C4 alkynyl or an alkyl group containing a substituent, e.g. hydroxyalkyl, haloalkyl, cyanoalkyl, carboxyalkyl, acylalkyl, oxyalkyl, sulfonylalkyl, sulfonylamidoalkyl, amidoalkyl, carbonoylalkyl, ureylalkyl, etc.

In the beta-secretase inhibitors of the invention a connecting moiety is bound to the chlorophenyl group consisting of a single bond, a C₁-C₄ alkylene group, a C₂-C₈ alkenylene group, a C₁-C₄ alkylene group containing a least one heteroatom or C₂-C₈ alkenylene group containing at least one heteratom, preferably 1 to 3, more preferably 1 to 2 heteroatoms. Preferably, the one or more heteroatoms are selected from N, O and S, more preferably from N and S. Most preferred are connecting moieties R² containing two N atoms. The connecting moiety R² is preferably a single bond, a —CH₂—S—, —CH═N—NR⁸—, —C(CH₃)═N—NR⁸—, —CH═N—CH₂—, —C(CH₃)═N—CH₂—, —CH═CH—NR⁸—, —C(CH₃)═CH—NR⁸—, —CH═N—O—, —C(CH₃)═N—O—, —CH═CH—S—, —C(CH₃)═CH—S—, —CH═CH—CH₂—, —C(CH₃)═CH—CH₂—, —CH═N—S—, —C(CH₃)═N—S—, —CH₂—NH—NH—, —C(CH₃)—NH—NH—, —CH₂—NH—CH₂—, —C(CH₃)—NH—CH₂—, —CH₂—CH₂—NH—, —C(CH₃)—CH₂—NH—, —CH₂—NH—O—, —C(CH₃)—NH—O—, —CH₂—CH₂—O—, —C(CH₃)—CH₂—O—, —CH₂—NH—S—, —C(CH₃)—NH—S—, —CH₂—CH₂—S—, —C(CH₃)—CH₂—S—, —CH₂—CH₂—CH₂—, —C(CH₃)—CH₂—CH₂—, —CH—N═N—, —C(CH₃)—N═N—, —CH═N⁺(CH₃)—NR⁸—, —C(CH₃)═N⁺(CH₃)—NR⁸—, —CH═N⁺(CH₃)—O—, —C(CH₃)═N⁺(CH₃)—O—, —CH═N⁺(CH₃)—S— or —C(CH₃)═N⁺(CH₃)—S— group.

R⁸ can be hydrogen or any group as stated herein for R⁴.

The connecting moiety R² connects the halophenyl residue, in particular, a chlorophenyl residue with a further cyclic moiety. Said second cycle can be a mono- or polycycle, in particular, a polycycle condensed from of two, three or four cycles. The cyclic moiety preferably contains one or more heteroatoms selected from O, N and S. Especially preferred examples of the Cyc group are

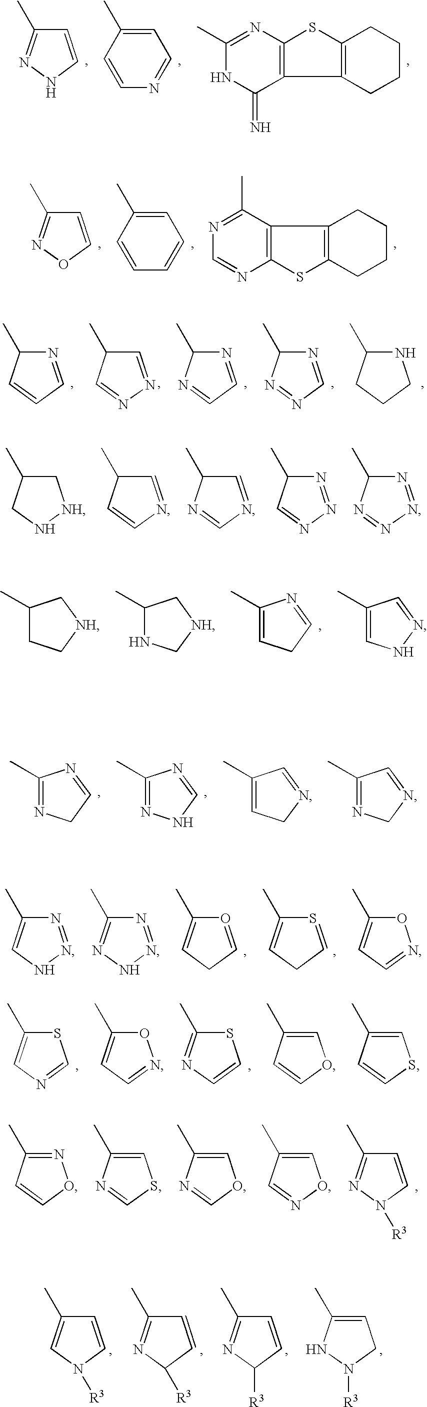

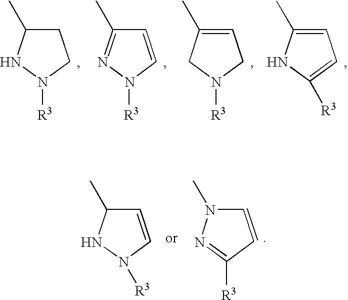

According to the invention the cyclic moiety Cyc again can be substituted with up to eight substituents, preferably up to five substituents. Examples of particularly preferred substituents on the cyclic moiety Cyc are Cl, N, methyl, allyl, paraiodophenyl, NO₂, CF₃ as well as

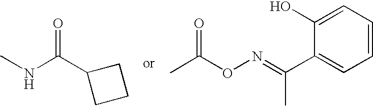

Most preferably, the beta-secretase inhibitor of the invention is selected from the following compounds:

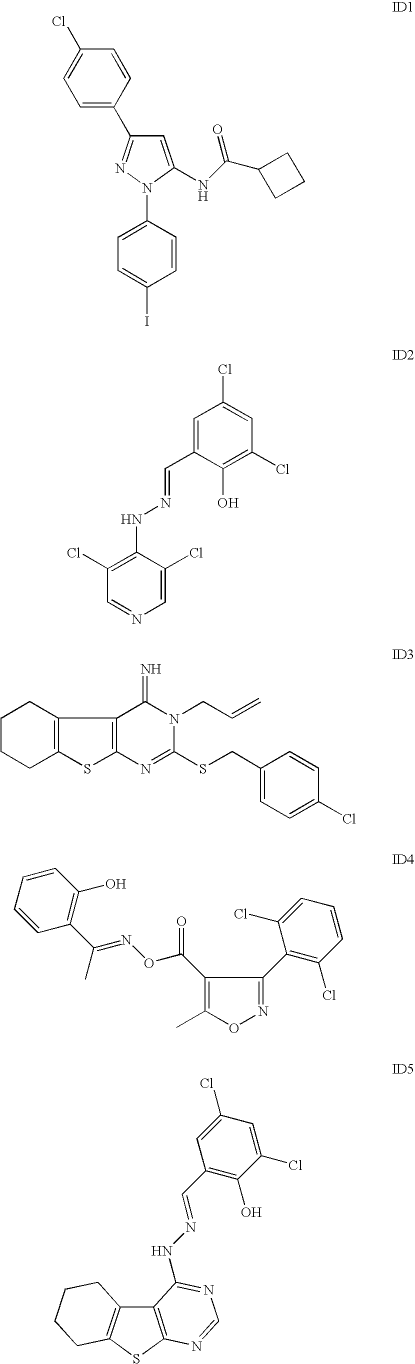

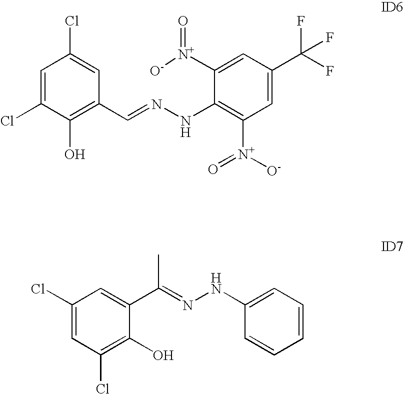

The terms used herein have the following meanings, unless stated otherwise.

The term “hydrocarbon” or “hydrocarbon group” comprises any moiety which contains at least one carbon atom and at least one hydrogen atom. In particular, the term “hydrocarbon” denotes any moiety having from 1 to 30 carbon atoms and includes aromatic and aliphatic groups.

The term “aliphatic” or “aliphatic group” means:
a straight chain that is completely saturated or that contains one or more units of unsaturation
a monocyclic C3-C8 hydrocarbon or bicyclic C8-C12 hydrocarbon that is completely saturated or that contains one or more units of unsaturation, but which is not aromatic (herein after referred to as “carbocyclic”), and that has a single connection point to the rest of the molecule. Any individual ring in the bicyclic system contains three to seven ring atoms.

Aliphatic groups include, but are not limited to, linear or branched or alkyl, alkenyl, alkynyl groups, carbocyclic groups (e.g. methyl, ethyl, n-propyl, butyl, isobutyl, sec-buytl, pentyl, acetyl, propionyl, butyrl, benzoyl, etc.) and hybrids thereof such as cycloalkyl-alkyl, cycloalkenyl-alkyl or cylcoalkyl-alkenyl (e.g. cylclpropyl, cyclobutyl, cyclopentyl, cyclohexyl, etc.). In each aliphatic group, up to 4 carbons may be independently replaced by O, N, S, or NH.

The terms “alkyl”, “alkenyl” or “alkynyl” used alone or as part of a larger moiety include both straight and branched chains, wherein up to 4 carbons may be independently replaced by O, N, S or NH. Unless otherwise stated the chain lengths of alkyl, alkenyl and alkynyl contains one to twelve carbon atoms and at least two carbon atoms and one double bond, in the case of alkenyl, and at least two carbon atoms and one triple bond, in the case of alkynyl.

The term “heteroatom” includes oxygen and any oxidized form of nitrogen and sulphur, and the quaternized form of any basic nitrogen.

The term “aryl” or “aryl ring” used alone or as part of a larger moiety as in “arylalkyl”, “arylalkoxy” or “aryloxyalkyl” refers to monocyclic, bicyclic or tricyclic ring systems having a total of five to fourteen ring members, wherein at least one ring in the system is aromatic and wherein each ring contains three to seven ring members (e.g. phenyl, naphtyl, tetrahydronaphtyl etc.)

The term “heterocycle”, “heterocyclic”, “heteroaryl”, “heteroaryl ring” and “heteroaromatic” alone or used in a larger moiety refers to monocyclic, bicyclic or tricyclic , saturated or unsaturated ring systems having a total of five to fourteen ring members, at least one ring in the system contains a heteroatom and wherein each ring contains three to seven ring members (e.g. pyridyl, triazolyl, benzthiazolyl, thienly, morphonlinyl, quinolyl, furyl, imidazolyl, pyrazinyl, pyrimidinyl, quinoxalinyl etc.)

The compounds of this invention may contain one or more “asymmetric” carbon atoms and thus may occur as racemates and racemic mixtures, single enantiomers, diastereomic mixtures or individual diastereomers. All such isomeric forms of these compounds are expressly included in the present invention. Each stereogenic carbon may be of R or S configuration. Although specific compounds and scaffolds exemplified in this invention may be depicted in a particular stereochemical configuration, compounds and scaffolds having either the opposite stereochemistry at any given chiral center or mixtures thereof are also envisaged.

The term “query” refers to a model or pattern which is used to search chemical compound databases to find chemical, biological and pharmacological compounds which are similar to this query.

The term “focused library” refers to a selection of a subset of compounds from a larger collection of chemical compounds. This can be done automatically by the use of computer methods using a query and an appropriate software tool or by manual selection of compounds.

The term “common pharmacophore” refers to the general pharmacophoric representation of the binding site of one or more distinct protein class or classes e.g. aspartyl proteases, phosphodiesterases or serine protease. The common pharmacophore combines pharmacophores of different ligands of protein belonging to one or more protein classes and represents a model or pattern for possible ligands or inhibitors of the distinct protein class or protein classes.

The term “Surf2Lead” refers to a method which uses three-dimensional protein information to extract two or three dimensional pharmacophoric information from a potential or known binding site of a protein (herein after referred to as “inverse active site”) (WO 02/92218 A2). The pharmacophore represents a model or a pattern to find new potential ligands or inhibitors for the specific or other similar proteins.

The term “PHACIR screening” refers to the use of binary patterns (herein after referred to as “binary fingerprints”) as queries to generate a focused library (WO 02/12889 A2). The binary fingerprints can be generated from two or three dimensional pharmacophores of one ore more known ligands or inhibitors or from one or more inverse active sites. By searching chemical compound databases this method leads to similar but new potential ligands or inhibitors.

The compounds of this invention may be prepared by general methods known to those skilled in the art (for further references see e.g. Houben-Weyl Methods in Organic Chemistry, 4^{th} ed). One having ordinary skill in the art may synthesize other compounds of this invention following the technique of specification using reagents that are readily synthesized or commercially available.

Particularly preferred compounds are:

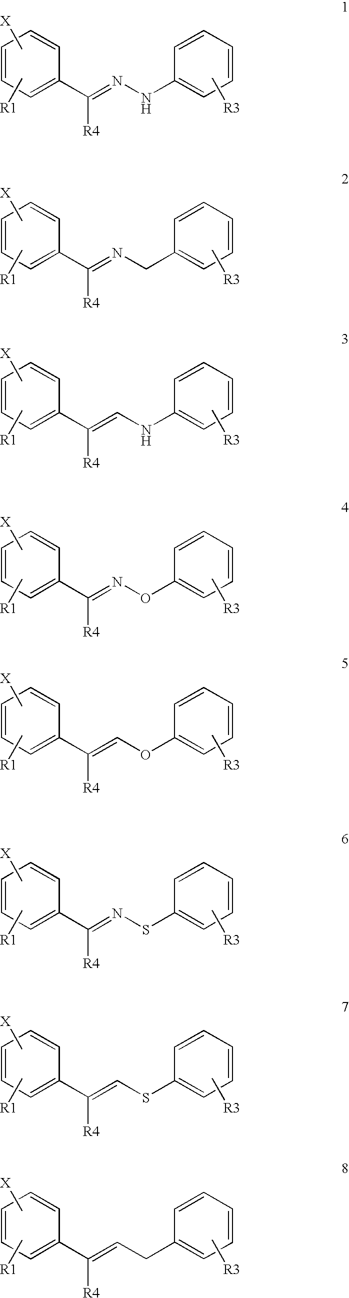

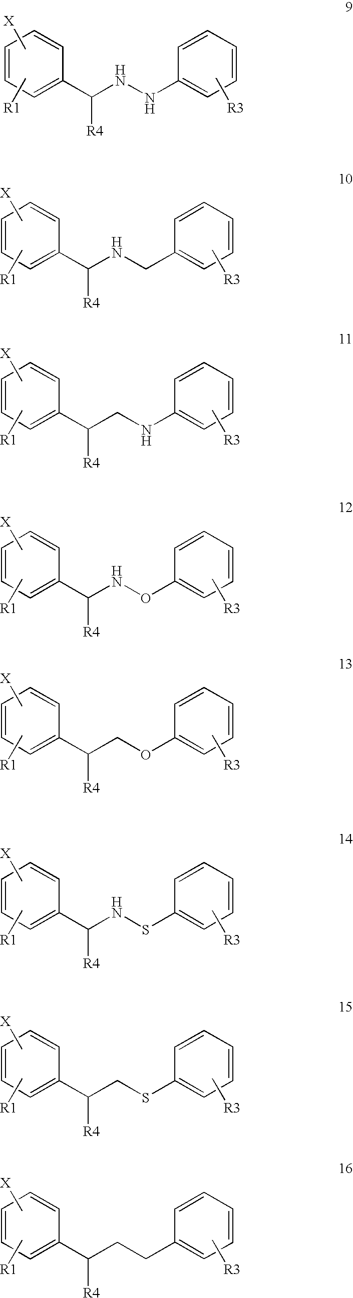

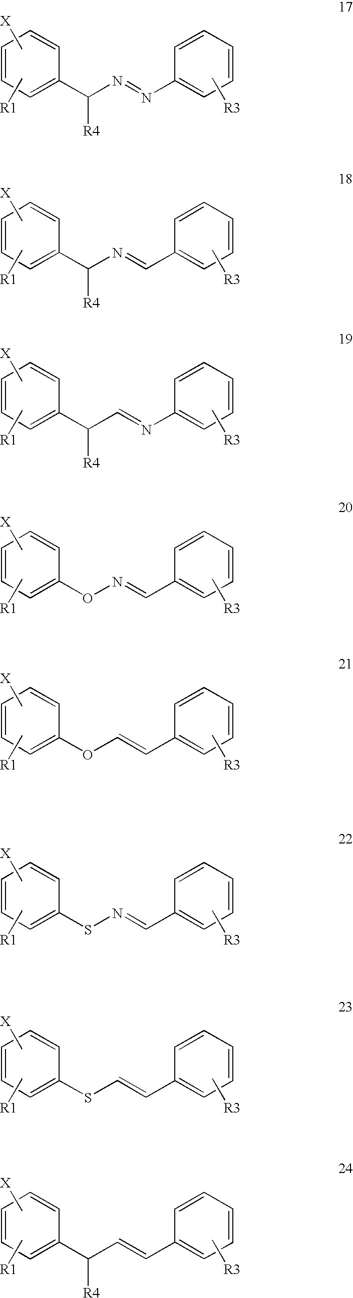

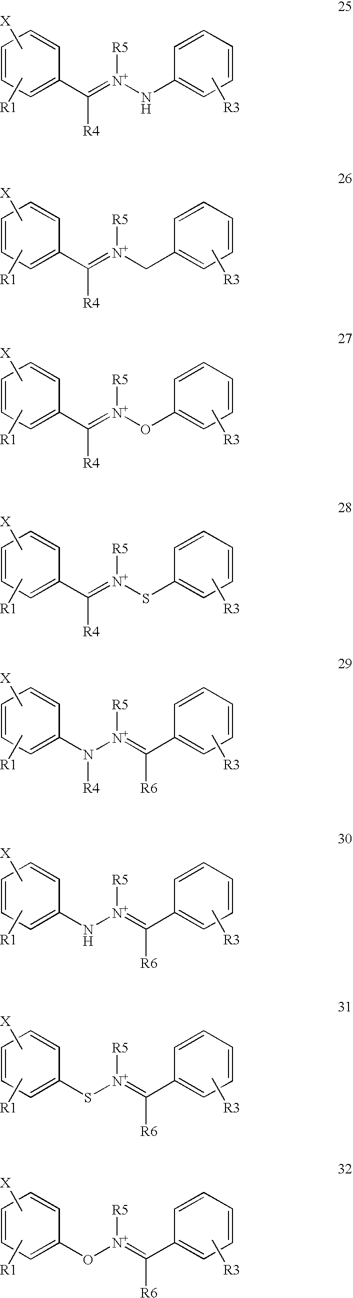

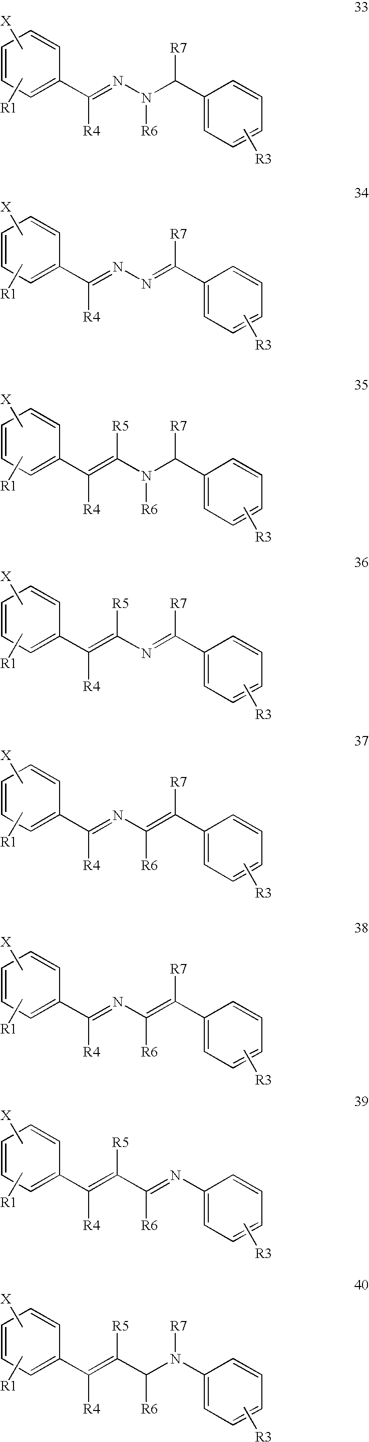

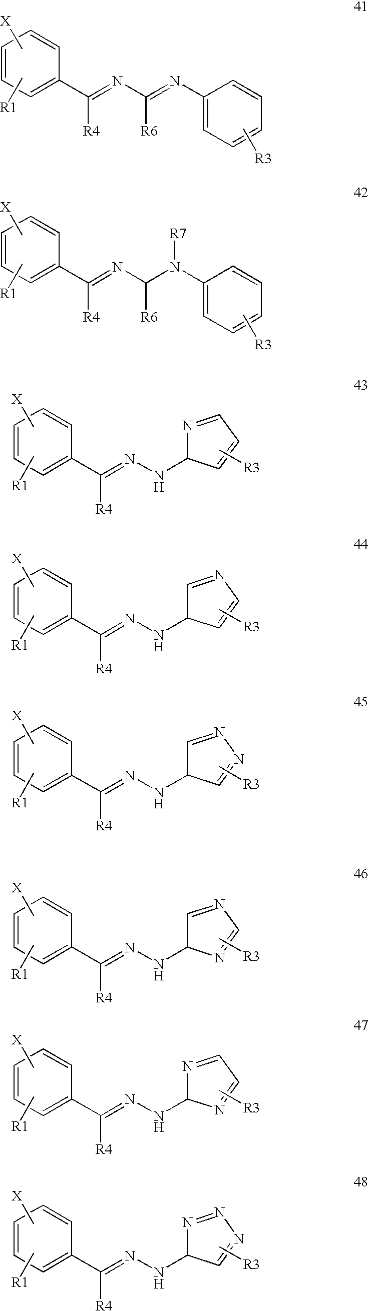

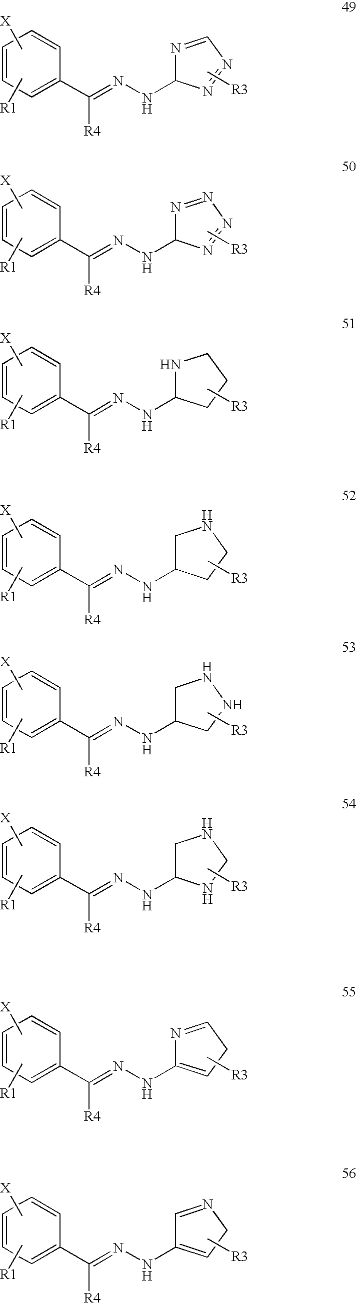

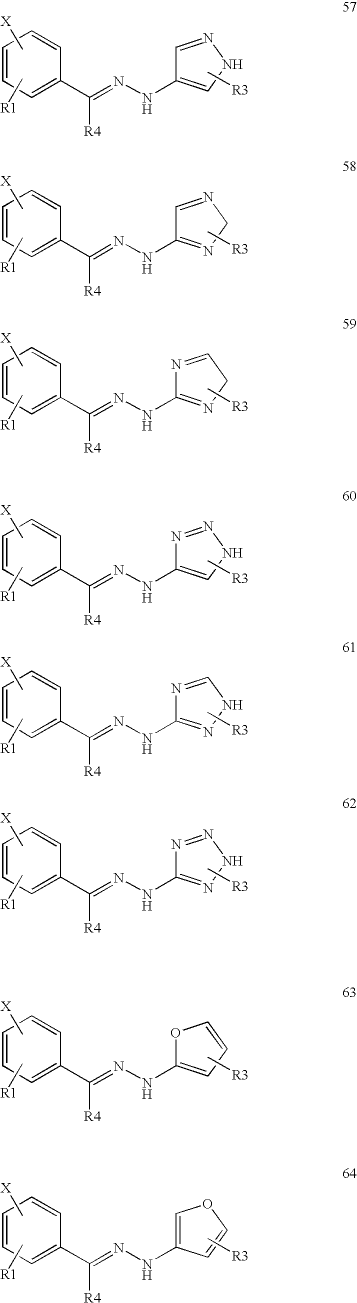

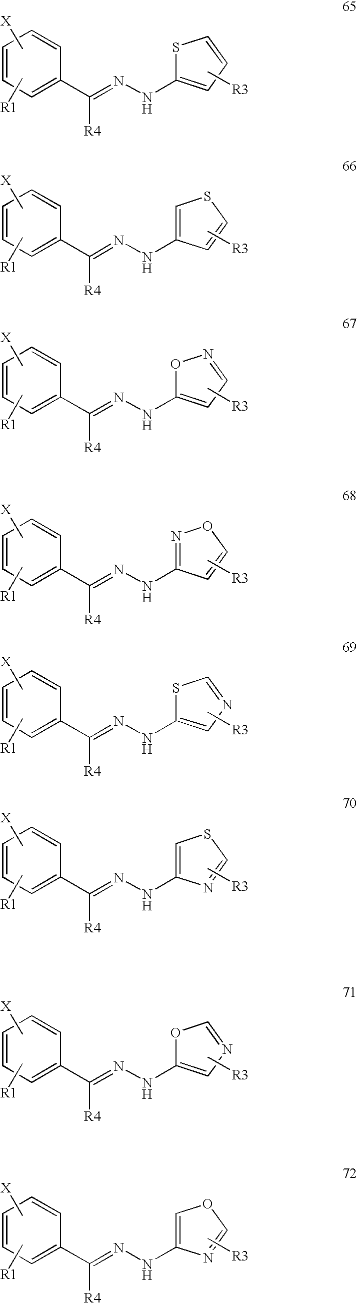

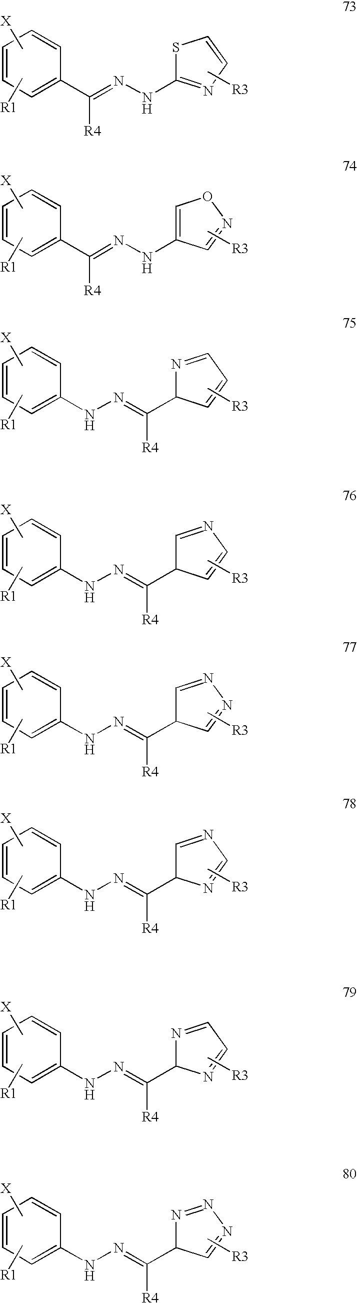

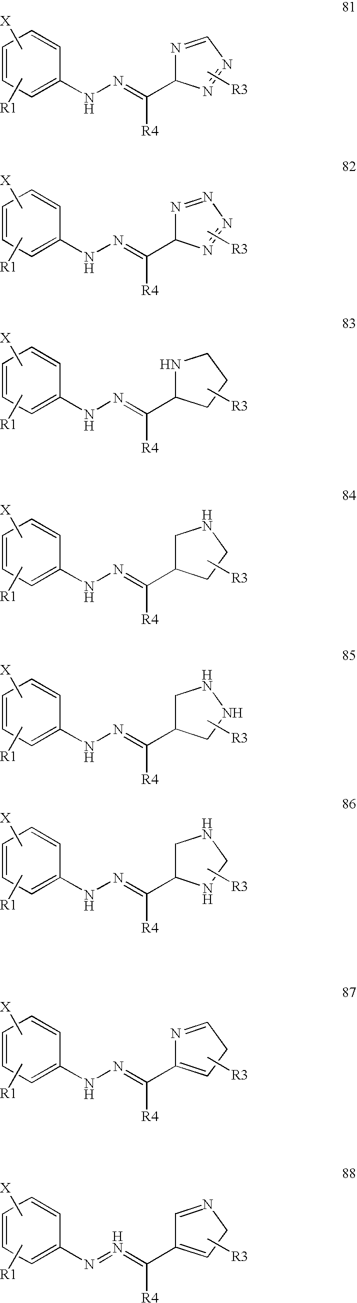

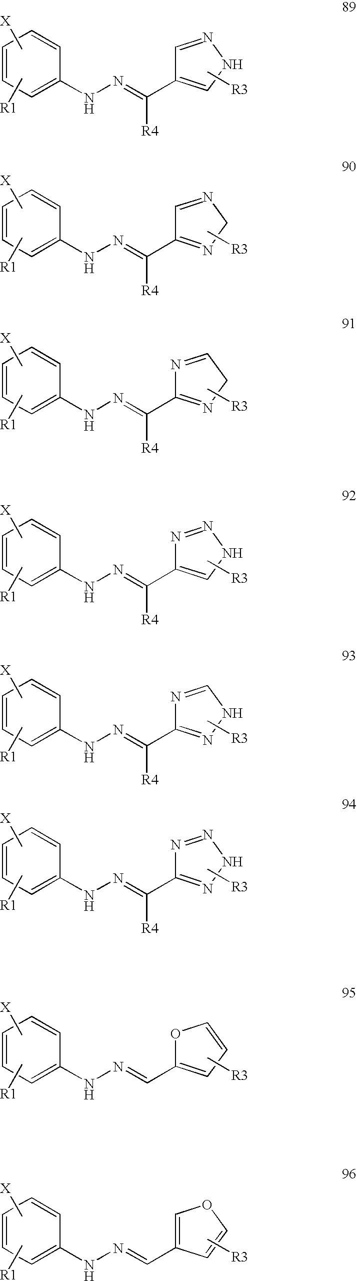

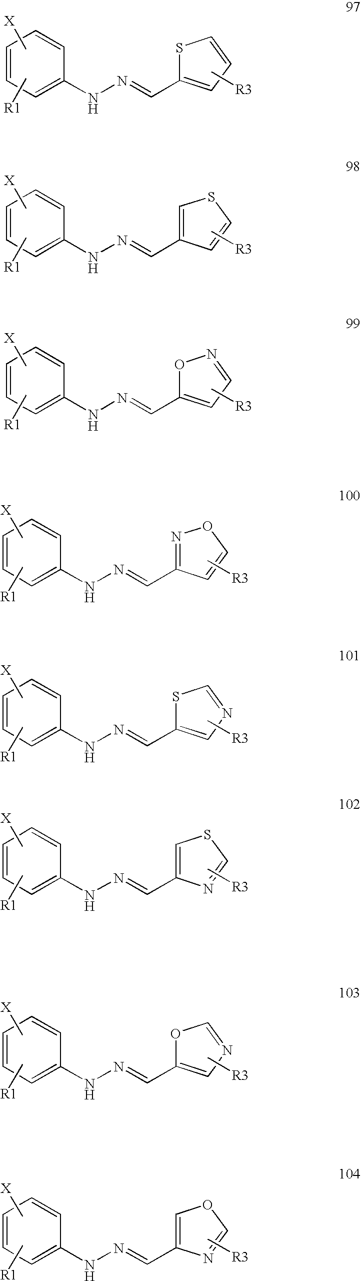

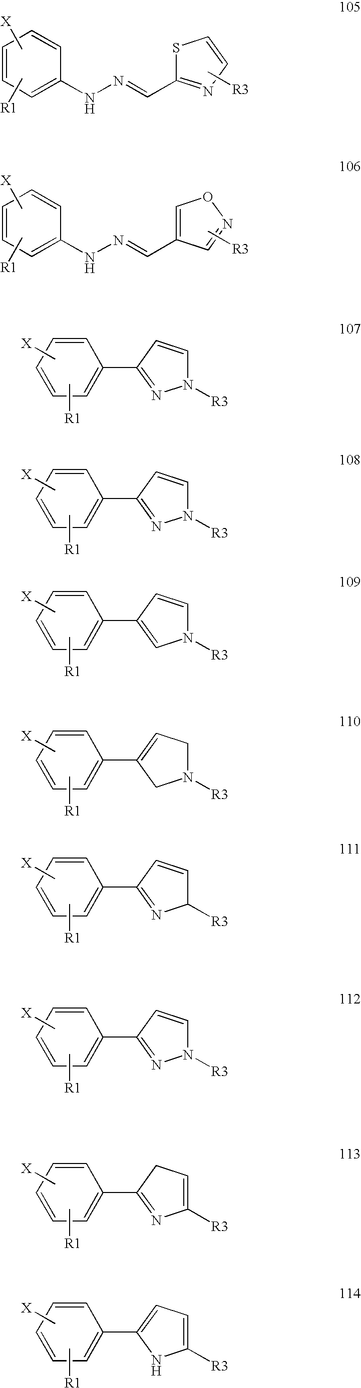

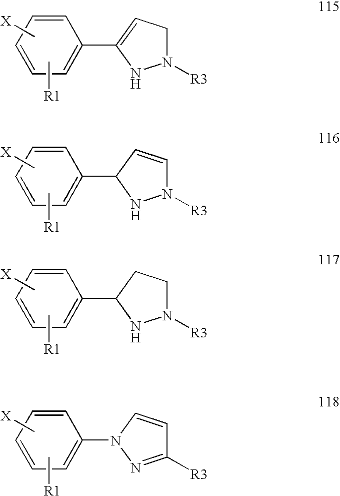

Each R4, R5, R6 and R7 independently represents halogen, hydroxy, cyano, trifluoromethyl, C1-C4 alkyl, C2-C4 alkenyl or C2-C4 alkynyl which may be substituted, e.g. hydroxyalkyl, haloalkyl, cyanoalkyl, carboxyalkyl, acylalkyl, oxyalkyl, sulfonylalkyl, sulfonylamidoalkyl, amidoalkyl, carbonoylalkyl, ureylalkyl, etc. or a moiety which is bioisosteric thereto.

The beta-secretase inhibitors of the invention are potent compounds, by means of which beta-secretase can be inhibited selectively and effectively. They are characterized, in particular, by IC50 values of ≦200 μM. Further, the compounds of the invention provide new scaffolds for the development of novel drugs based on beta-secretase inhibitors.

The compounds of the invention are further characterized in that they are active in cells. In this context, compounds ID3 and ID7 are particularly preferred because these are especially cell-permeable active compounds.

The compounds of the invention were identified by applying computerized screening, especially PHACIR screening, for the generation of a focused library out of a compound data base based on a combined pharmacophore. In this way it is possible to discover beta-secretase inhibitors having new structures, which had not yet been presumed in the art to have such activity.

As combined pharmacophore, for example, a combination of common pharmacophore for aspartyl proteases and a surface-based (surf2lead®) pharmacophore of the crystallized beta-secretase:OM922 complex can be used. For the common pharmacophore of the aspartyl proteases the active center was employed for generation of the pharmacophore. For the surf2lead approach the surface of the active center of the beta-secretase: OM922 complex crystallized with inhibitor was used for generation of the pharmacophore. A query for PHACIR screening was generated from a combination of the two pharmacophores. The compounds of the focused library identified by virtual screening then can be subjected to an in vitro assay, e.g. a fluorescence BACE assay, or a cellular assay in order to determine its possible inhibitory action.

As described above, compounds having beta-secretase inhibitory action are suitable agents for the treatment of Alzheimer's disease and other disorders characterized by beta A deposits like Down's Syndrome and HCHWA-D. The invention therefore also relates to a pharmaceutical composition comprising a beta-secretase inhibitor as described above, optionally in admixture with one or more pharmaceutically acceptable carriers, diluents and/or excipients.

The compounds of the invention are particularly suited to inhibit the formation of beta amyloid peptides from the amyloid precursor protein (APP). Thus, any condition or disease can be treated which is caused by a pathological accumulation of beta amyloid such as Alzheimer's disease, Trisomy 21 (Down's Syndrome) or Hereditary Cerebral Hemorrhage with Amyloidosis of the Dutch type (HCHWA-D).

The pharmaceutical composition can be formulated for administration according to the respective demands. In particular, it can be formulated for topical, oral, transdermal, parenteral, sublingual, intranasal, intrathecal, rectal, inhalative or intravenous administration.

For oral delivery suitable administration forms include e.g. tablets, pills, troches, gel or capsules. For parenteral delivery e.g. administration by depot, syringe, ampoule or vial can be employed. Formulations in the form of pathces, medipad, ointments or creams are suitable for topical delivery.

The amount of inventive inhibitor required for administration in the treatment and/or prophylaxis of a disease such as Alzheimer's disease depends on the seriousness of the condition as well as on the patient to be treated. Typically, a daily dose is 0.01 mg/kg of body weight to 500 mg/kg of body weight, preferably at least 0.1 mg/kg of body weight to 50 mg/kg of body weight.

Besides the beta-secretase inhibitor the pharmaceutical compositions of the invention can contain one or more other active substances.

The invention further relates to the use of a beta-secretase inhibitor as described above for the manufacture of a drug for the treatment of diseases which are mediated by beta-secretase. The beta-secretase inhibitors are especially suited for the production of a drug for the treatment of Alzheimer's disease. The expression “treatment of a condition” as used herein refers both to the treatment of established symptoms and a prophylactic treatment, by which the occurrence of the disease or particular symptoms can be avoided.

The invention further relates to a substance library containing at least 5, preferably at least 10, more preferably at least 50 compounds as described therein. Such library can be used especially for screening in activity tests.

The invention is further illustrated by the following Example.

### EXAMPLE 1

Fluorescence BACE Assay

The inhibitory activity of the compounds of the invention was shown in an in vitro assay, namely a fluorescence BACE assay.

The assay was set up in triplicate wells of 96 well black plate. rhBACE was diluted to 1 unit/well in 100 I (PBS +0.5% Triton-X 100, pH 5). BACE enzyme (obtained from R&D systems (ca.No. 931-AS), reference: Vasser et al., 1999, Science 286, 735-741) was incubated with various concentrations of inhibitor compound (10 nM to 500 M) for 5 min; Reaction was started by adding peptide substrate (obtained from BACHEM (cat. No. M-2470), reference: Ermolieff et al., Biochemistry 39 (2000) 12450-56) with EDANS/Dabcyl labels. After incubation for 2 hours at 37 C the results were read in fluoroplate reader at 355 nm/486 nm.

The following IC50 values were determined for the above-mentioned particularly preferred compounds:
ID1: IC50=45 μM; ID2: IC50=29 μM; ID3: IC50=10 μM; ID4: IC50=140−170 μM; ID5: IC50=53 μM; ID6: IC50=39.3 μM and ID7: IC50=14.4 μM

1. Beta-secretase inhibitor of formula (1) 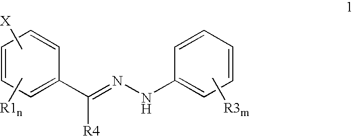
wherein
X: represents a halogen or a moiety which is bioisosteric thereto, in particular, F, Cl, Br, I, Methyl or CF₃, preferably Cl;
R1: each independently represents halogen; hydroxy, cyano, trifluoromethyl, nitro, a hydrocarbon group containing 1 to 4 carbon atoms, in particular, C1-C4 alkyl, C2-C4 alkenyl or C2-C4 alkynyl, which may be substituted, e.g. hydroxyalkyl, haloalkyl, cyanoalkyl, carboxyalkyl, acylalkyl, oxyalkyl, sulfonylalkyl, sulfonylamidoalkyl, amidoalkyl, carbonoylalkyl, ureylalkyl, etc. or a moiety which is bioisosteric thereto and n=0 to 2.
R3: each independently selected from R1 or is a aryl or heterocyclic moiety substituted by 0 to 4 moieties from R1 or a group selected from 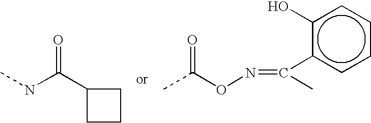
R4: represents halogen, hydroxy, cyano, trifluoromethyl, C1-C4 alkyl, C2-C4 alkenyl or C2-C4 alkynyl which may be substituted, e.g. hydroxyalkyl, haloalkyl, cyanoalkyl, carboxyalkyl, acylalkyl, oxyalkyl, sulfonylalkyl, sulfonylamidoalkyl, amidoalkyl, carbonylalkyl, ureylalkyl, etc. or a moiety which is biosteric thereto.
and m=0 to 4.

2. Beta-secretase inhibitor according to claim 1 having the formula

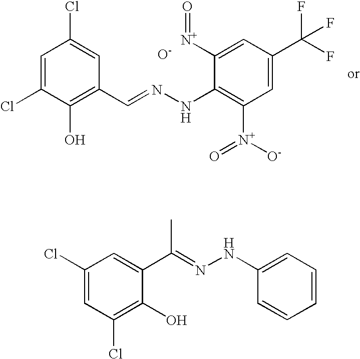

3. Beta-secretase inhibitor according to claim 1 or 2, having an IC50≦200 μM.

4. Beta-secretase inhibitor according to any of claims 1 to 3, being active in cells.

5. A pharmaceutical composition comprising a beta-secretase inhibitor according to any of claims 1 to 4,
optionally in admixture with one or more pharmaceutically acceptable carriers, diluents and/or excipients.

6. A substance library containing at least 5 beta-secretase inhibitors according to any of claims 1 to 4.

7. The use of a beta-secretase inhibitor according to any of claims 1 to 4 for the manufacture of a pharmaceutical agent for the treatment or prevention of a condition which is mediated by beta-secretase.

8. The use of a beta-secretase inhibitor according to any of claims 1 to 4 for the manufacture of a pharmaceutical agent to inhibit the formation of beta amyloid peptides from the amyloid precursor protein (APP).

9. The use according to claim 7 or 8 for the manufacture of a pharmaceutical agent for the treatment or prevention of Alzheimer's disease or any disorder,caused by pathological deposits of beta amyloid peptides.

10. Use of a beta-secretase inhibitor according to any of claims 1 to 4 in the manufacture of a pharmaceutical, agent for the treatment or prevention of conditions selected from the group consisting of Alzheimer's disease, Down syndrome, cerebral amyloid angiopathy, Hereditary Cerebral Hemorrhage with Amyloidosis of the Dutch type (HCHWA-D) and other degenerative dementia characterized by beta-amyloid deposits.

11. A method of treating or preventing a disease characterized by beta-amyloid deposits such as Alzheimer's disease by modulating the activity of the beta-amyloid converting enzyme, comprising administering to a patient in need of such treatment a compound according to claims 1 to 4, or a pharmaceutically acceptable salt thereof.

12. Beta-secretase inhibitor having the formula [chemical structure]
